(19) United States
(12) Patent Application Publication
(10) Pub. No.: US 2016/0015708 A1
(43) Pub. Date: Jan. 21, 2016

(54) STABLE DOSAGE FORMS OF IMATINIB MESYLATE

(71) Applicant: RANBAXY LABORATORIES LIMITED, New Delhi, Delhi (IN)

(72) Inventors: Ravi KOCHHAR, Gurgaon (IN); Annavarapu RAMESH, Hyderabad (IN); Suneel Kumar VASIREDDY, Khammam (IN); Roshan Lal SANDAL, Firozpur (IN)

(21) Appl. No.: 14/379,681

(22) PCT Filed: Feb. 15, 2013
(86) PCT No.: PCT/IB2013/051261

§ 371 Date: Aug. 19, 2014

(30) Foreign Application Priority Data
Feb. 21, 2012 (IN) 498/DEL/2012
Feb. 21, 2012 (IN) 499/DEL/2012

(51) Int. Cl. A61K 31/506; A61K 9/48; B65D 81/26; A61K 9/28; A61J 1/03; A61K 9/20; A61K 9/16

(52) U.S. Cl. CPC A61K 31/506; A61K 9/20; A61K 9/48; A61K 9/2095; A61K 9/4833; A61K 9/1688; A61K 9/2893; A61J 1/035; B65D 81/264

(57) ABSTRACT

The present invention relates to a stable oral pharmaceutical dosage form comprising imatinib mesylate and one or more pharmaceutically acceptable excipients wherein the amount of imatinib calculated as free base is more than 80% by weight based on the total weight of the oral pharmaceutical dosage form, and wherein the oral dosage form does not show polymorphic conversion after storage at 40° C. and 75% relative humidity for three months. It also relates to processes for the preparation thereof.

### FIELD OF THE INVENTION

The present invention relates to a stable oral pharmaceutical dosage form comprising imatinib mesylate and one or more pharmaceutically acceptable excipients, wherein the amount of imatinib calculated as free base is more than 80% by weight based on the total weight of the oral pharmaceutical dosage form, and wherein the oral dosage form does not show polymorphic conversion after storage at 40° C. and 75% relative humidity for three months. It also relates to processes for the preparation thereof.

### BACKGROUND OF THE INVENTION

Imatinib is indicated for the treatment of non-malignant and malignant proliferative disorders such as chronic myelogeneous leukemia (CML), gastrointestinal stromal tumors (GIST), and other conditions. Currently, it is marketed in United States by Novartis under the trade name Gleevec® as film coated tablets containing imatinib mesylate equivalent to 100 mg or 400 mg of imatinib free base. Imatinib mesylate is designated chemically as 4-[(4-methyl-1-piperazinyl)methyl]-N-[4-methyl-3-[[4-(3-pyridinyl)-2-pyrimidinyl]amino]-phenyl]benzamide methanesulfonate. Gleevec® capsules, 50 mg and 100 mg, were the subject of NDA 21335, held by Novartis Pharmaceutical Corp., and was initially approved on May 10, 2001.

U.S. Pat. No. 5,521,184 discloses imatinib, processes for its preparation, and its use, especially as an antitumor agent.

WO Publication No. 2003/090720 discloses a tablet comprising a pharmacologically effective amount of imatinib, or a pharmaceutically acceptable salt thereof, in an amount from about 30% to 80% by weight of the active moiety based on the total weight of the tablet.

U.S. Pat. No. 6,894,051 describes the alpha and the beta crystalline forms of imatinib mesylate. U.S. Pat. No. 7,544,799 discloses a crystalline form of imatinib mesylate having non-needle-shaped crystals.

WO Publication No. 2009/042803 describes a pharmaceutical composition comprising imatinib mesylate in an amount of about 23% to 29% w/w of the total composition. Further, WO Publication No. 2009/042809 discloses a pharmaceutical composition comprising an initial polymorphic form of imatinib mesylate, wherein less than 10% of the polymorphic form of imatinib mesylate is converted to Form α or Form β after storage at 40° C. at 75% relative humidity for one month. WO Publication No. 01/47507 exemplifies a pharmaceutical composition or tablet containing about 22% w/w of imatinib mesylate. Also, U.S. Publication Nos. 2006/0275372 and 2009/0136579 describe nanoparticulate compositions of imatinib.

WO Publication No. 2011/121593 exemplifies film coated tablets comprising imatinib mesylate in an amount of 90% to 97% based on total weight of the coated tablet, where the tablets are coated with a film coating comprising polyvinyl alcohol applied to the tablet core in an amount of 1% to 2% w/w of the tablet.

U.S. Pat. No. 6,958,335 describes the use of imatinib or a pharmaceutically acceptable salt thereof in the treatment of gastrointestinal stromal tumors (GIST).

WO Publication No. 2005/077933 discloses pharmaceutical compositions comprising imatinib mesylate α₂-Form in the range of 45% to 60% w/w, wherein the excipients used are selected from microcrystalline cellulose, lactose, crospovidone XL, colloidal silicon dioxide, magnesium stearate, talc, or mixtures thereof.

Imatinib mesylate is typically prescribed in high doses, e.g., 400 mg to 800 mg daily as a treatment of leukemia in adults. Imatinib is generally known to be a hygroscopic material. Thus, in view of the high dosage needed for the therapy and the nature of the active ingredient, there is a further need to optimize the pharmaceutical dosage forms comprising imatinib mesylate so that it is convenient to manufacture, administer, and simultaneously provide the requisite daily dosage of imatinib. It is also required that the said pharmaceutical dosage forms exhibit polymorphic stability upon storage, as this is essential to ensure an adequate shelf life of the final dosage form. It is therefore an objective of the present invention to develop an optimized stable pharmaceutical dosage form that contains a high dosage amount of imatinib mesylate and also exhibits polymorphic stability throughout the shelf life.

### SUMMARY OF THE INVENTION

In one general aspect, the present invention relates to a stable oral pharmaceutical dosage form comprising imatinib mesylate and one or more pharmaceutically acceptable excipients, wherein the amount of imatinib calculated as free base is more than 80% by weight based on the total weight of the oral pharmaceutical dosage form, and wherein the oral pharmaceutical dosage form does not show polymorphic conversion after storage at 40° C. and 75% relative humidity for three months.

In an embodiment of the above aspect, the pharmaceutically acceptable excipients are selected from diluents, binders, disintegrants, lubricants, glidants, granulating solvents, and coloring agents.

In another embodiment, imatinib mesylate is present in alpha crystalline form.

In yet another embodiment, the alpha crystalline form does not convert to the beta crystalline form when the oral pharmaceutical dosage form is stored at 40° C. and 75% relative humidity for three months.

In another embodiment, the stable oral pharmaceutical dosage form is a tablet.

In another embodiment, the stable oral pharmaceutical dosage form is a capsule.

In a further embodiment, the stable oral pharmaceutical dosage form is dispensed in a package comprising blister packs or high-density polyethylene (HDPE) bottles.

In a further embodiment, the package may additionally contain a desiccant.

In another general aspect, the present invention relates to a process for preparing a stable oral pharmaceutical dosage form comprising imatinib mesylate and one or more pharmaceutically acceptable excipients, wherein the amount of imatinib calculated as free base is more than 80% by weight based on the total weight of the oral pharmaceutical dosage form, and wherein the oral dosage form does not show polymorphic conversion after storage at 40° C. and 75% relative humidity for three months, and wherein the process comprises the conventional processes of dry granulation or wet granulation.

### DETAILED DESCRIPTION OF THE INVENTION

The present invention provides stable oral pharmaceutical dosage forms comprising imatinib mesylate in a polymorphic form, for example, alpha crystalline form, and one or more pharmaceutically acceptable excipients such that the amount of imatinib calculated as free base is more than 80% by weight based on the total tablet weight.

The stable oral pharmaceutical dosage forms can be tablets or capsules.

As used herein, the term “pharmaceutically acceptable excipient” includes conventional pharmaceutical additives known in the art such as diluents, binders, disintegrants, lubricants, glidants, granulating solvents, coloring agents, or combinations thereof.

Preferred diluents include microcrystalline cellulose, silicified microcrystalline cellulose, microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, and the like.

Preferred binders include polyvinylpyrrolidone, cross-linked polyvinylpyrrolidone, cellulose gums (e.g., carboxymethyl cellulose, hydroxypropyl methylcellulose, and hydroxypropyl cellulose), pregelatinized starch, acacia, guar gum, alginic acid, carbomer, dextrin, maltodextrin, and the like.

Preferred disintegrants include mannitol, alginic acid, carboxymethylcellulose, hydroxypropylcellulose, microcrystalline cellulose, croscarmellose sodium, povidone, crospovidone, magnesium aluminum silicate, methylcellulose, sodium alginate, starches or modified starches such as sodium starch glycolate, corn starch, potato starch or pregelatinized starch, and the like.

Preferred lubricants include magnesium stearate, zinc stearate, calcium stearate, stearic acid, sodium stearyl fumarate, vegetable oil, mineral oil, and the like.

Preferred glidants include talc, colloidal silicon dioxide, corn starch, and the like.

Preferred granulating solvents include water, ethanol, methanol, isopropyl alcohol, methylene chloride, acetone, and the like.

Suitable coloring agents include those approved for use by the United States Food and Drug Administration (FDA), such as iron oxide, and are well known to those skilled in the art.

The tablets or capsules may be prepared by conventional processes, for example, by dry granulation or wet granulation.

When the pharmaceutical dosage form is a tablet, it may further be coated using conventional coating techniques. The tablets may be coated with one of the commercially available coating systems such as Opadry® or any polymeric film coating routinely used in the formulation of pharmaceutical compositions such as ethyl cellulose, hydroxypropylmethylcellulose, hydroxypropylcellulose, methylcellulose, carboxymethyl cellulose, hydroxyl methylcellulose, cellulose acetate, waxes such as polyethylene glycol, methacrylic acid polymers, and the like.

The term “stable”, as recited herein, refers to the polymorphic stability of imatinib mesylate in the oral pharmaceutical dosage form which implies that imatinib mesylate remains in its initial polymorphic form, i.e., alpha crystalline form without undergoing polymorphic conversion to the beta crystalline form, for example, upon storage at 40° C. and 75% relative humidity during the period of three months. Several factors may affect the stability of the active ingredient, for example, the type of coating material used may influence the stability of the drug and hence may contribute towards the shelf life of the final tablet dosage form. Besides this, the type of packaging may also contribute to the stability of the drug. The packaging material may be comprised of high-density polyethylene bottle (HDPE) bottles, various types of blister packs, or similar pharmaceutically acceptable packaging material. The package may additionally contain a desiccant. A desiccant is any drying agent that removes moisture from the air. Desiccants include, but are not limited to, silica gel, clay desiccants, calcium sulfate, calcium chloride, calcium oxide, zeolite, activated alumina, activated charcoal, and the like.

The stable oral pharmaceutical dosage forms comprising imatinib mesylate as described herein may take the form of several different embodiments.

In one embodiment, the stable oral pharmaceutical dosage forms is a tablet which comprises the alpha crystalline form of imatinib mesylate and one or more pharmaceutically acceptable excipients, such that the amount of imatinib calculated as free base is about 82% by weight based on the total tablet weight. The tablet may then be coated with a commercially available Opadry® dispersion.

In another embodiment, the stable oral pharmaceutical dosage form is a tablet which comprises the alpha crystalline form of imatinib mesylate and one or more pharmaceutically acceptable excipients, such that the amount of imatinib calculated as free base is about 83% by weight based on the total tablet weight.

In both of the above embodiments, the tablets may be dispensed in packaging made with usual packaging materials such as HDPE bottles or blister packs.

In one embodiment, the tablet may be prepared by:
a. granulating imatinib mesylate alone or mixed with one or more pharmaceutically acceptable excipients with a granulating solvent;
b. drying and sizing the granules of step (a);
c. optionally, mixing with one or more pharmaceutically acceptable excipients;
d. compressing the resultant blend into tablets using appropriate tooling; and
e. optionally, coating the tablets with a coating.

In another embodiment, the tablet may be prepared by:
a. compacting imatinib mesylate alone or mixed with one or more pharmaceutically acceptable excipients by slugging;
b. milling and sizing the slugs into granules;
c. optionally, mixing the granules with one or more pharmaceutically acceptable excipients;
d. compressing the resultant blend into tablets using appropriate tooling; and
e. optionally, coating the tablets with a coating.

In another embodiment, the stable oral pharmaceutical dosage form is a capsule which comprises alpha crystalline form of imatinib mesylate and one or more pharmaceutically acceptable excipients, such that the amount of imatinib calculated as free base is about 83% by weight based on the total capsule fill weight.

In the above embodiment, the said capsule may be dispensed in packs made with usual packaging materials such as HDPE bottles or blister packs.

In one embodiment, the capsules may be prepared by:
a. granulating imatinib mesylate alone or mixed with one or more pharmaceutically acceptable excipients with a granulating solvent;
b. drying and sizing the granules of step (a);
c. optionally, mixing with one or more pharmaceutically acceptable excipients; and
d. filling the resultant blend into suitable sized capsules.

In another embodiment, the capsules may be prepared by:
a. granulating imatinib mesylate with a mixture of isopropyl alcohol and water;
b. drying and sizing the granules of step (a);
c. mixing the granules of step (b) with magnesium stearate; and
d. filling the resultant blend into suitable sized capsules.

From the above, it is apparent that various modifications and combinations of the formulations detailed in the text may be made without departing from the spirit and scope of the invention. The invention as described herein may be illustrated by the following examples but is not to be construed to be limited by them.

### EXAMPLES

Example 1

| | | Quantity in mg/tablet | |
|---|---|---|---|
| | | | Comparative |
| S/N | Ingredients | Example 1 | Example 1 |

Intragranular

| | | | |
|---|---|---|---|
| 1. | Imatinib mesylate (alpha form) | 478.0 | 478.0 |

Granulating solvent

| | | | |
|---|---|---|---|
| 2. | Ispropyl alcohol:purified water | q.s. | q.s. |

Extragranular

| | | | |
|---|---|---|---|
| 3. | Magnesium stearate | 2.0 | 2.0 |

| | | |
|---|---|---|
| Tablet Weight | 480.0 | 480.0 |

Coating

| | | | |
|---|---|---|---|
| 4. | Opadry ® brown | 9.0 | — |
| 5. | Opadry ® clear | — | 4.0 |
| 6. | Purified water | q.s. | q.s. |

| | | |
|---|---|---|
| Coated Tablet Weight | 489.0 | 484.0 |

### Procedure:

Imatinib mesylate was granulated with a mixture of isopropyl alcohol and purified water. The granules obtained were dried and sized. The resultant granules were blended with magnesium stearate and compressed into tablets. The obtained tablets were coated with Opadry® brown aqueous dispersion in purified water in Example 1 and Opadry® clear aqueous dispersion in purified water in Comparative Example 1.

The above-prepared tablets were dispensed in suitable packs. To further assess the polymorphic stability, these were subjected to accelerated stability testing at 40° C./75% relative humidity for three months. The percentage of beta crystalline form of imatinib mesylate was determined during and after three months interval. It was found that the tablets coated with Opadry® brown were stable while the tablets coated with Opadry® clear were unstable and showed polymorphic conversion. The results of the stability studies are summarized in Table 1 below.

TABLE 1
Percentage of beta crystalline form of imatinib mesylate in tablets prepared as per the above Example
1 and Comparative Example 1 when stored at 40° C./75% relative humidity in respective packs

| | | Percentage of beta crystalline form of imatinib mesylate | | | | | | | |
|---|---|---|---|---|---|---|---|---|---|
| | Time | | | | | | | | |
| | Initial | 1 month | | | | 3 months | | | |
| | Pack | | | | | | | | |
| | | | | HDPE | HDPE | | | HDPE | HDPE |
| | | | | Bottle without | Bottle with | | | Bottle without | Bottle with |
| | | CFB | DCFB | desiccant | desiccant | CFB | DCFB | desiccant | desiccant |
| Example 1 | ND | ND | — | 0.97 | — | ND | ND | 58.03 | ND |
| Comparative | ND | D | — | — | — | 106.6 | — | 106.57 | — |
| Example 1 | | | | | | | | | |

Meanings of abbreviations: D—Detectable, ND—Not detectable, HDPE—High-density polyethylene, CFB—Cold form blister, DCFB—Desiccant embedded cold form blister.
(—) symbolizes that data was not available/generated at the time.

### Examples 2-6

| | | Quantity in mg/tablet | | | | |
|---|---|---|---|---|---|---|
| | | Examples | | | | |
| S/N | Ingredients | 2 | 3 | 4 | 5 | 6 |

Intragranular

| | | | | | | |
|---|---|---|---|---|---|---|
| 1. | Imatinib mesylate (alpha | 478.0 | 478.0 | 478.0 | 478.0 | 478.0 |
| | form) | | | | | |
| 2. | Iron oxide yellow | — | — | — | — | 0.488 |
| 3. | Iron oxide red | — | — | — | — | 0.488 |

Granulating solvent

| | | | | | | |
|---|---|---|---|---|---|---|
| 4. | Isopropyl alcohol:purified | q.s. | — | — | — | q.s. |
| | water | | | | | |
| 5. | Isopropyl alcohol | — | q.s. | — | — | — |
| 6. | Purified water | — | — | q.s. | — | — |

Extragranular

| | | | | | | |
|---|---|---|---|---|---|---|
| 7. | Polyplasdone XL | — | — | — | — | 3.264 |
| 8. | Magnesium stearate | 2.0 | 2.0 | 2.0 | 2.0 | 1.76 |

| | | | | | |
|---|---|---|---|---|---|
| Tablet Weight | 480.0 | 480.0 | 480.0 | 480.0 | 484.0 |

Coating

| | | | | | | |
|---|---|---|---|---|---|---|
| 9. | Opadry ® brown | 9.0 | 9.0 | 9.0 | 9.0 | — |
| 10. | Purified water | — | — | q.s. | — | — |
| 11. | Isopropyl alcohol:meth- | q.s. | q.s. | — | q.s. | — |
| | ylene chloride | | | | | |

| | | | | |
|---|---|---|---|---|
| Coated Tablet Weight | 489.0 | 489.0 | 489.0 | 489.0 |

### Procedure:

### Example 2

Imatinib mesylate was granulated with a mixture of isopropyl alcohol and purified water. The granules obtained were dried and sized. The resultant granules were blended with magnesium stearate and compressed into tablets. The obtained tablets were coated with Opadry® brown non-aqueous dispersion in a mixture of isopropyl alcohol and methylene chloride.

### Example 3

Imatinib mesylate was granulated with isopropyl alcohol. The granules obtained were dried and sized. The resultant granules were blended with magnesium stearate and compressed into tablets. The obtained tablets were coated with Opadry® brown non-aqueous dispersion in a mixture of isopropyl alcohol and methylene chloride.

### Example 4

Imatinib mesylate was granulated with purified water. The granules obtained were dried and sized. The resultant granules were blended with magnesium stearate and compressed into tablets. The obtained tablets were coated with Opadry® brown aqueous dispersion in purified water.

### Example 5

Imatinib mesylate was compacted by slugging. The slugs obtained were milled and sized into granules. The resultant granules were blended with magnesium stearate and compressed into tablets. The obtained tablets were coated with Opadry® brown non-aqueous dispersion in a mixture of isopropyl alcohol and methylene chloride.

### Example 6

Imatinib mesylate, iron oxide yellow, and iron oxide red were granulated with a mixture of isopropyl alcohol and purified water. The granules obtained were dried and sized. The resultant granules were blended with polyplasdone XL and magnesium stearate and compressed into tablets.

The above tablets of Examples 1-6 were dispensed in several kinds of packs. To further assess the polymorphic stability, these were subjected to accelerated stability testing at 40° C./75% relative humidity for three months. The percentage of beta crystalline form of imatinib mesylate was determined during and after three months interval. The results of the stability studies are summarized in Table 2 below.

TABLE 2
Percentage of beta crystalline form of imatinib in tablets prepared as per above
Examples 2-6 when stored at 40° C./75% relative humidity in respective packs

| | | Percentage of beta crystalline form of imatinib mesylate | | | | | | | |
|---|---|---|---|---|---|---|---|---|---|
| | Time | | | | | | | | |
| | Initial | 1 month | | | | 3 months | | | |
| | Pack | | | | | | | | |
| | | | | HDPE | HDPE | | | HDPE | HDPE |
| | | | | Bottle without | Bottle with | | | Bottle without | Bottle with |
| | | CFB | DCFB | desiccant | desiccant | CFB | DCFB | desiccant | desiccant |
| Example 2 | ND | ND | ND | 42.21 | ND | ND | ND | D | ND |
| Example 3 | ND | — | ND | — | ND | — | ND | — | ND |
| Example 4 | ND | D | ND | D | ND | — | — | — | — |
| Example 5 | ND | — | ND | — | ND | — | ND | — | ND |
| Example 6 | ND | — | — | — | — | ND | — | 7.35 | — |

Meanings of abbreviations: D—Detectable, ND—Not detectable, HDPE—High-density polyethylene, CFB—Cold form blister, DCFB—Desiccant embedded cold form blister.
(—) symbolizes that data was not available/generated at the time.

### Examples 7-8

Quantity in mg/capsule

| | | | |
|---|---|---|---|
| S/N | Ingredients | Example 7 | Example 8 |

Intragranular

| | | | |
|---|---|---|---|
| 1. | Imatinib mesylate (alpha form) | 478.0 | 119.5 |

Granulating Solvent

| | | | |
|---|---|---|---|
| 2. | Isopropyl alcohol:purified water | q.s. | q.s. |

Extragranular

| | | | |
|---|---|---|---|
| 3. | Magnesium stearate | 2.0 | 0.5 |

| | | |
|---|---|---|
| Capsules Fill Weight | 480.0 | 120.0 |

### Procedure:

Imatinib mesylate was granulated with a mixture of isopropyl alcohol and purified water. The granules obtained were dried and sized. The resultant granules were blended with magnesium stearate and filled into suitable sized capsules.

The above prepared capsules were dispensed in suitable packs. To assess the polymorphic stability, these capsules were subjected to accelerated stability testing at 40° C./75% relative humidity for three months. The percentage of beta crystalline form of imatinib mesylate was determined during and after three months interval. The results clearly indicated that the capsules were stable even on storage for a period of three months. The results of the stability studies are summarized in Table 3 below.

TABLE 3
Percentage of beta crystalline form of imatinib mesylate
in capsules prepared as per the above Examples 7-8 when stored
at 40° C./75% relative humidity in respective packs

| | | Percentage of beta crystalline form of imatinib mesylate | | | | | |
|---|---|---|---|---|---|---|---|
| | Time | | | | | | |
| | Initial | 1 month | | 2 months | | 3 months | |
| | Pack | | | | | | |
| | | | HDPE | | HDPE | | HDPE |
| | | | Bottle without | | Bottle without | | Bottle without |
| | | CFB | desiccant | CFB | desiccant | CFB | desiccant |
| Example 7 | ND | ND | — | ND | — | ND | ND |
| Example 8 | ND | ND | — | ND | — | ND | ND |

Meanings of abbreviations: ND—Not detectable, HDPE—High-density polyethylene, CFB—Cold form blister, (—) symbolizes that data was not available/generated at the time.

1. A stable oral pharmaceutical dosage form comprising imatinib mesylate in alpha crystalline form and one or more pharmaceutically acceptable excipients, wherein the amount of imatinib calculated as free base is more than 80% by weight based on the total weight of the oral pharmaceutical dosage form, wherein said oral pharmaceutical dosage form is dispensed in a package selected from blister pack, or high-density polyethylene (HDPE) bottle, and wherein the alpha crystalline form does not convert to beta crystalline form when the said oral pharmaceutical dosage form is stored at 40° C. and 75% relative humidity for three months.

2. The stable oral pharmaceutical dosage form according to claim 1, wherein the pharmaceutically acceptable excipients are selected from diluents, binders, disintegrants, lubricants, glidants, granulating solvents, and coloring agents.

3. The stable oral pharmaceutical dosage form according to claim 1, wherein the stable oral pharmaceutical dosage form is a tablet.

4. The stable oral pharmaceutical dosage form according to claim 1, wherein the stable oral pharmaceutical dosage form is a capsule.

5. (canceled)

6. The stable oral pharmaceutical dosage form according to claim 1, wherein the package may additionally contain a desiccant.

7. A process for preparing the stable oral pharmaceutical dosage form of claim 3 comprising:
a. granulating imatinib mesylate alone or mixed with one or more pharmaceutically acceptable excipients with a granulating solvent;
b. drying and sizing the granules of step (a);
c. optionally, mixing with one or more pharmaceutically acceptable excipients;
d. compressing the resultant blend into tablets using appropriate tooling; and
e. optionally, coating the tablets with a coating.

8. A process for preparing the stable oral pharmaceutical dosage form of claim 3 comprising:
a. compacting imatinib mesylate alone or mixed with one or more pharmaceutically acceptable excipients by slugging;
b. milling and sizing the slugs into granules;
c. optionally, mixing the granules with one or more pharmaceutically acceptable excipients;
d. compressing the resultant blend into tablets using appropriate tooling; and
e. optionally, coating the tablets with a coating.

9. A process for preparing the stable oral pharmaceutical dosage form of claim 4 comprising:
a. granulating imatinib mesylate alone or mixed with one or more pharmaceutically acceptable excipients with a granulating solvent;
b. drying and sizing the granules of step (a);
c. optionally, mixing with one or more pharmaceutically acceptable excipients; and
d. filling the resultant blend into suitable sized capsules.
